(19) United States
(12) Patent Application Publication
(10) Pub. No.: US 2023/0182053 A1
(43) Pub. Date: Jun. 15, 2023

(54) AIR PURIFIER

(71) Applicant: ACCO Brands Corporation, Lake Zurich, IL (US)

(72) Inventors: Bryan J. Poteracki, McHenry, IL (US); Michael Graban, Hoffman Estates, IL (US); Adam Himmelspach, Spring Grove, IL (US); Andrew Monteleone, Chicago, IL (US)

(21) Appl. No.: 18/077,667

(22) Filed: Dec. 8, 2022

Related U.S. Application Data
(60) Provisional application No. 63/361,379, filed on Dec. 15, 2021.

(51) Int. Cl. B01D 46/00; B01D 46/24; A61L 9/20

(52) U.S. Cl. CPC B01D 46/0005; A61L 9/20; B01D 46/0004; B01D 46/0028; B01D 46/2403; A61L 2209/12; A61L 2209/14; B01D 2265/028; B01D 2279/40

(57) ABSTRACT

An air purifier having a side-access or horizontally-removable air filter assembly may include a housing, a base plate, a filter plate, a filter, and an air blowing unit. In some configurations the air purifier also includes a UV-light source and a removable covering or panel. Once the removable cover is separated from the housing, the filter plate with the filter assembly may be slid horizontally into and out of the housing. Once the filter plate, filter, and UV-light source are removed from the housing, the filter may be replaced, cleaned, and/or inspected.

### CROSS-REFERENCE TO RELATED APPLICATION

This application claims the benefit of U.S. Provisional Application No. 63/361,379, filed on Dec. 15, 2021, which is hereby incorporated by reference in its entirety.

### TECHNICAL FIELD

This disclosure relates generally to air purifiers, and more specifically, to air purifiers that may have a removable air filter.

### BACKGROUND

Air purifiers help remove contaminants such as particulates and/or impurities from ambient air. Air purifiers often use air filters to remove some of the contaminants from the air. Air filters may collect and store these contaminants on the surface thereof or throughout the filter. Over time, use of such air filters typically causes air purifiers to become less effective. Accordingly, manufacturers of air purifiers often recommend that air filters should be regularly removed from the air purifier unit and either cleaned or replaced.

Removing the air filter and either replacing or cleaning the filter impacts the effectiveness of the air filter, and in turn the air purifier. Removing and replacing the air filter may require partial deconstruction of the air purifier unit. One known method for removing the air filter may include sliding the air filter upward out from or over a base of the unit when a portion of the outer shell has been removed. Another known method is to invert the unit and slide the air filter out of the bottom of the unit after removing a filter cover. To facilitate easy, regular maintenance, removal of air filters from air purifiers should be quick and convenient for users. Current designs, especially for high performing filters, tend to be overly complicated for users and less than intuitive.

For example, some air purifiers include an irradiation element, which is employed to expose ambient air and/or the air filter to radiation. These irradiation elements commonly are placed adjacent to the air filter to allow for the radiation to reach the air filter. Air purifiers containing an irradiation element and removable air filter may become particularly tedious to maintain in light of the additional obstructions incorporated into the unit.

### BRIEF DESCRIPTION OF THE DRAWINGS

Disclosed herein are embodiments of systems and apparatuses pertaining to providing a horizontally insertable air filter. This description includes drawings, wherein:

FIG. 1 is a top perspective view of an air purifier with a horizontal insertable air filter in accordance with various embodiments.

FIG. 2A is a top perspective view of the air purifier of FIG. 1 with a removable panel removed.

FIG. 2B is an expanded view of a portion of the air purifier of FIG. 1.

FIG. 3A is a top perspective view of an air purifier housing with a filter plate removed from the housing.

FIG. 3B is a top perspective view of an air purifier housing with a filter plate partially inserted into the housing.

FIG. 4A is an exploded view of the air purifier of FIG. 1 with portions removed therefrom.

FIG. 4B is an exploded view of the air purifier of FIG. 1 with an air filter raised about the filter plate.

FIG. 5A is a bottom perspective view of a filter plate in accordance with various embodiments.

FIG. 5B is a top perspective view of a filter plate in accordance with various embodiments.

FIG. 6 is a top perspective view of a base plate in accordance with various embodiments.

FIG. 7 is a top perspective view of an air filter in accordance with various embodiments.

FIG. 8 is a detailed partial lower cross-sectional view of a spring-loaded filter plate in accordance with various embodiments.

FIG. 9A is a side view of an air purifier with the air filter in an unseated position and with a removable panel removed therefrom in accordance with various embodiments.

FIG. 9B is a side view of the air purifier of FIG. 9A with the air filter in a seated position.

FIG. 9C is a front cross-sectional view of the air purifier of FIG. 9A with the air filter in a seated position.

FIG. 10 is a view of an air purifier in transit in accordance with various embodiments.

FIG. 11A is a top view of a filter plate in accordance with various embodiments.

FIG. 11B is a partial view of FIG. 11A showing the electric connection in accordance with various embodiments.

FIG. 12A is a front view of an air purifier having a rear housing column in accordance with various embodiments.

FIG. 12B is a partial view of the housing electrical connection in accordance with various embodiments.

FIG. 13 is a side perspective view of an air purifier with a horizontal insertable air filter in accordance with various embodiments.

FIG. 14 is a side perspective view of the air purifier of FIG. 13 with a removable panel separated from a remainder thereof.

FIG. 15 is a front perspective view of the air purifier of FIG. 13 with a removable panel removed from a remainder of thereof.

FIG. 16 is a top perspective view of an annular filter in accordance with various embodiments.

FIG. 17 is a front perspective view of the air purifier of FIG. 13 with a removable panel and filter removed.

FIG. 18 is a front perspective view of the air purifier of FIG. 13 with a removable panel, filter, and bulb shield removed from a remainder thereof.

FIG. 19 is a top perspective view of a filter plate in accordance with various embodiments.

FIG. 20 is a rear perspective view of a removable panel in accordance with various embodiments.

FIG. 21 is a bottom perspective view of a filter plate in accordance with various embodiments.

FIG. 22 is a top perspective view of a base plate in accordance with various embodiments.

FIG. 23 is a detailed partial lower cross-sectional view of a spring-loaded filter plate in accordance with various embodiments.

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various embodiments. Also, common but well-understood elements that are useful or necessary in a commercially feasible embodiment are often not depicted to facilitate a less obstructed view of these various embodiments. It will further be appreciated that certain actions and/or steps may be described or depicted in a particular order of occurrence while those skilled in the art will understand that such specificity with respect to sequence is not actually required. It will also be understood that the terms and expressions used herein have the ordinary technical meaning as is accorded to such terms and expressions by persons skilled in the technical field as set forth above except where different specific meanings have otherwise been set forth herein.

### DETAILED DESCRIPTION

Generally speaking, pursuant to these various embodiments and systems described herein which may be used to provide an air purifier with an easily removable and replaceable air filter. In one illustrative approach, the air purifier includes an air filter that is horizontally insertable in order to provide replacement and/or cleaning of the air filter. In some embodiments a keyed connection is utilized between a filter plate and a base plate to provide an air filter assembly for an easy removal and insertion feature. In some embodiments a friction fit is utilized between the filter plate and the base plate to provide an air filter assembly with an easy removal and insertion feature. In addition, the filter plate may include a biasing mechanism that facilitates a substantially airtight seal between the air filter and portions of the housing unit. In addition, an irradiation element may be provided. In some configurations, the irradiation element may be provided on or supported by the filter plate and/or a mounting plate, such that the irradiation element is able to decontaminate at least a portion of the surface of the air filter, and may be removed from the interior of the purifier housing with the filter plate when the filter plate is removed from the housing. In some configurations the side-loading or horizontal insertion permits the changing or replacement of the filter (and/or the irradiation element or light source) without having to overturn or otherwise tilt the air purifier from the stable, resting configuration. These and other benefits may become clearer upon making a thorough review and study of the following detailed description.

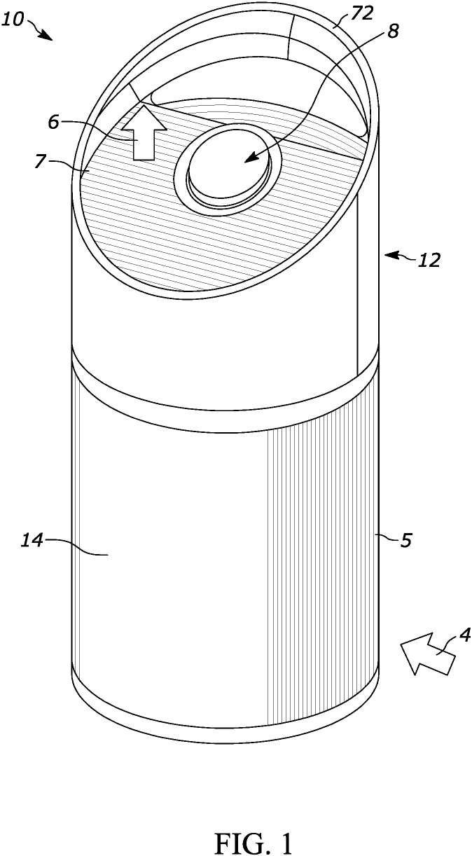

Referring now to the drawings, and in particular to FIG. 1, an illustrative embodiment of an air purifier 10 is provided in accordance with some embodiments. The air purifier 10 operates by drawing air in through an air intake 5 (as illustrated by arrow 4) on a side of the air purifier 10. The air purifier 10 may process the air through a housing 12 (which contains an air filter assembly) and expels substantially purified air (as illustrated by arrow 6) at an air outlet 7 on a top of the air purifier 10. The air purifier 10 may also include a user interface 8 and a unit handle 72 that a user may grab to manipulate or move the air purifier 10.

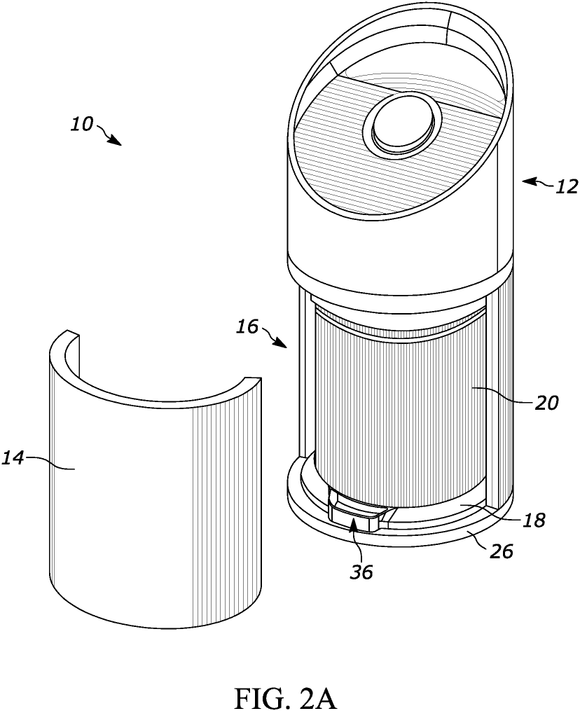

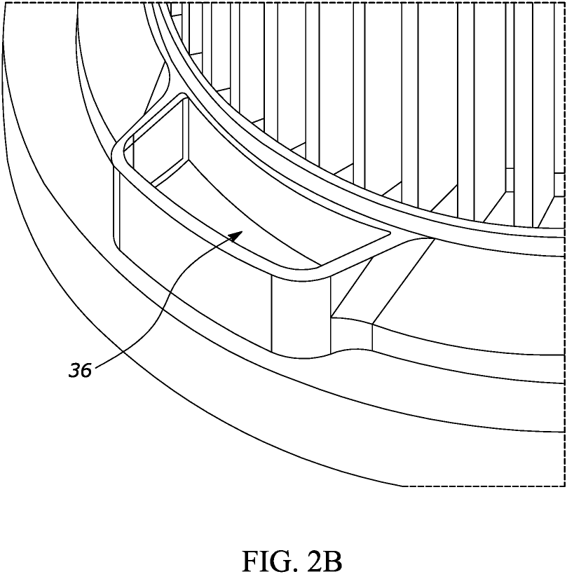

As shown in FIG. 2A, the air purifier 10 includes a housing 12 and a removable cover 14. An air blowing unit (as is known in the art) may be disposed within the housing 12. In one exemplary embodiment, the air purifier 10 includes a base plate 26 and a filter assembly 16. The filter assembly 16, described in more detail below, is removably disposed within the housing 12. In some embodiments, the filter assembly 16 includes a filter plate 18, an annular filter 20, and one or more ultra-violet (UV) light sources 32 removably retained to the filter plate 18. The annular filter 20 is removably disposed around the ultra-violet light sources 32, also referred to as the UV source or UV light source. The filter plate 18 may further include a handle 36, described in more detail below.

The annular filter 20 and the UV light source(s) 32 may be removed from the filter plate 18 and filter assembly 16 and replaced, cleaned, maintained, repaired, or otherwise accessible as needed.

The ultra-violet light source 32, also referred to as a UV light source, may be used to irradiate a surface of the annular filter 20 and air that is drawn into and through the annular filter 20. Irradiating the surface of the annular filter 20 may kill or otherwise inactivate contaminants retained or captured by the annual filter 20. Irradiating the surface of the annual filter 20 may extend the usable life span of the filter.

In some illustrative configurations, the UV light source 32 may include cathode tubes or bulbs (such as, e.g., hot, or cold cathode bulbs) or one or more light emitting diodes (LEDs), among other options. By some configurations, the UV light source 32 may include one, two, or more bulbs disposed near the center of the filter plate 18. In addition, as noted above, the UV light source 32 may be removed from the interior of the housing 12 and removed or replaced from the filter plate 18 and inserted into the interior of the housing 12.

In other illustrative configurations, the UV light source 32 may include discrete LEDs and utilize a system to directly irradiate the annular filter 20. In other illustrative embodiments a system to indirectly irradiate the annular filter 20 may utilize reflectors to redirect light from the UV light source 32 to irradiate the annular filter 20. In yet another configuration, the UV light source 32 may both directly and indirectly via one or more reflective surfaces irradiate the annular filter 20 and/or the air passing through the air purifier.

In further illustrative embodiments, the UV light source 32 may include one or more discrete LEDs in a tube, pipe, or some other transparent or opaque structure. The tube may act as a light tube to evenly distribute or diffuse the UV light across the annular filter 20, thus creating a more uniform irradiation of the annular filter 20. In other embodiments, an array of LEDs may be used to provide uniform irradiation of the annular filter 20. In some embodiments, the LEDs may be arranged in an array on a substrate, such as a printed circuit board (PCB).

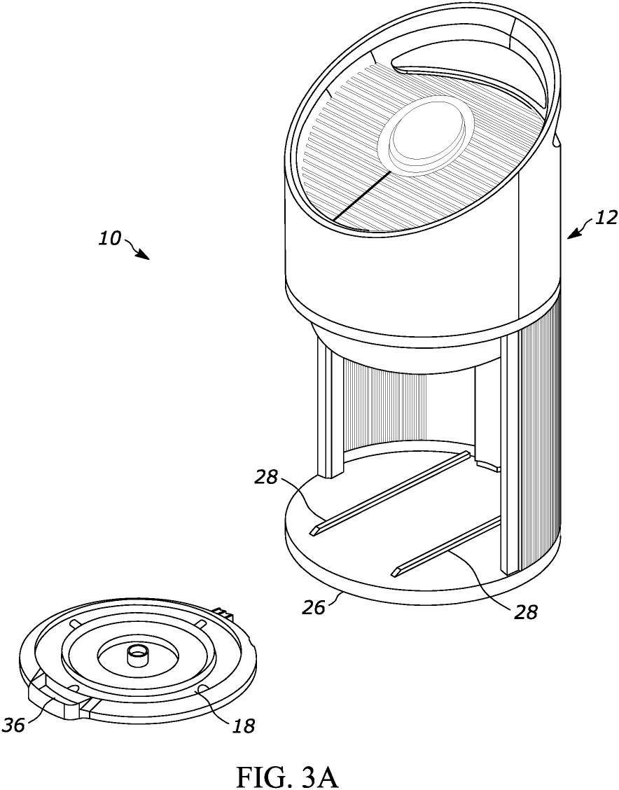

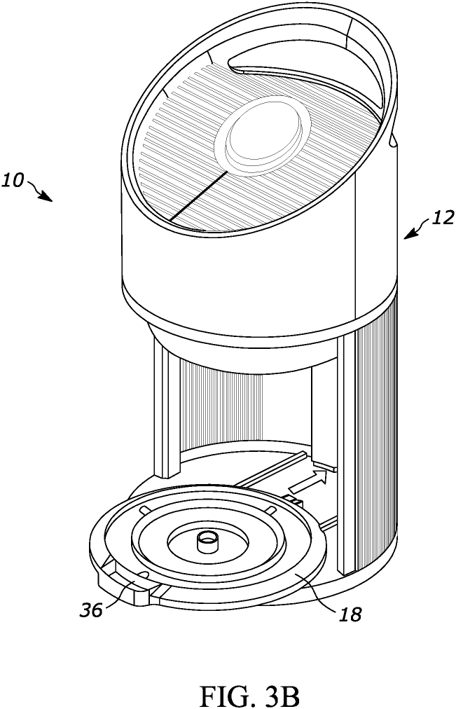

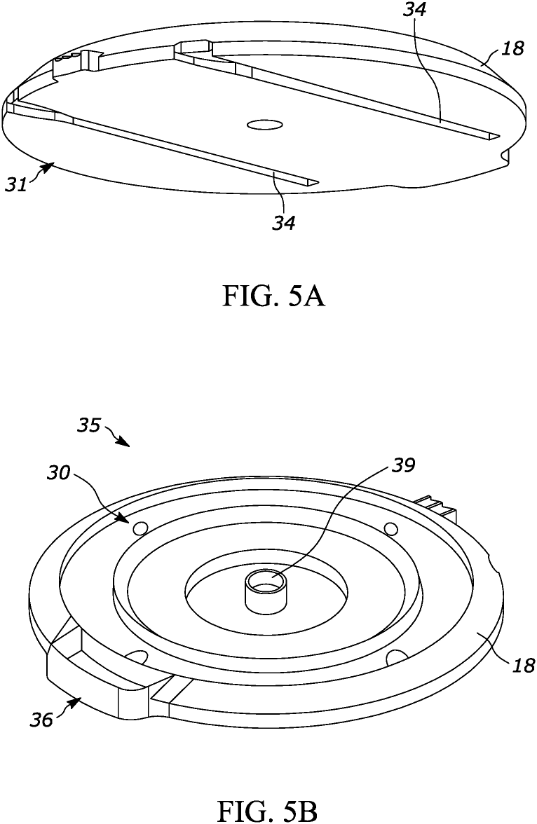

As shown illustratively in FIGS. 3A and 3B, the filter plate 18 is configured to slidably engage with the base plate 26 of the housing 12. This allows for the filter plate 18 and the entire filter assembly 16 to be easily moved into and out of the housing 12. This slidable engagement, which may be facilitated by a keyed connection, also may aid in aligning the filter plate 18, and related filter assembly 16, within the housing 12, which assist with proper engagement of the various electrical connections as discussed below. As shown in FIGS. 5A and 5B, the filter plate 18 has a top surface 35 and a bottom surface 31. The bottom surface 31 of the filter plate 18 and an upper surface 37 of the base plate 26 form the sliding connection, also referred to as a keyed connection. The filter plate 18 may also include a handle 36 for slidably engaging and disengaging the filter plate 18, and its corresponding keyed connection, from the base plate 26.

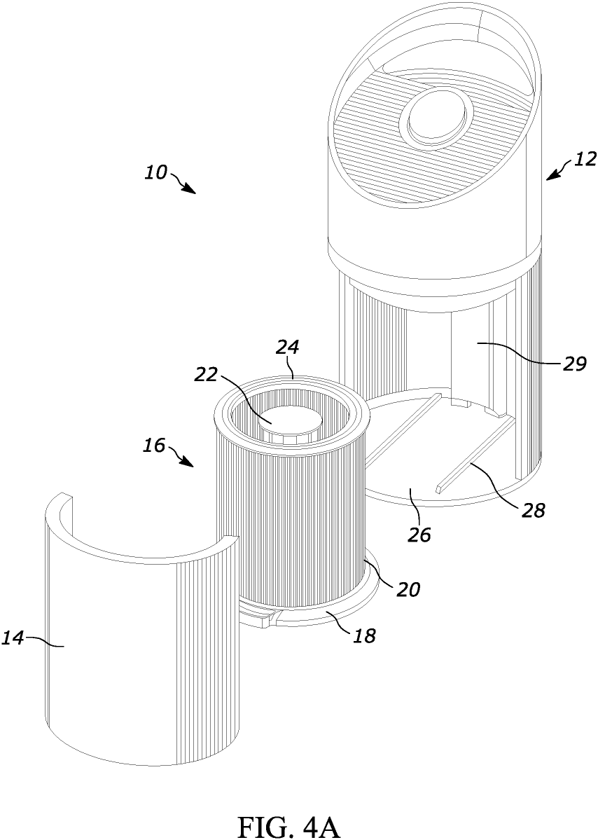

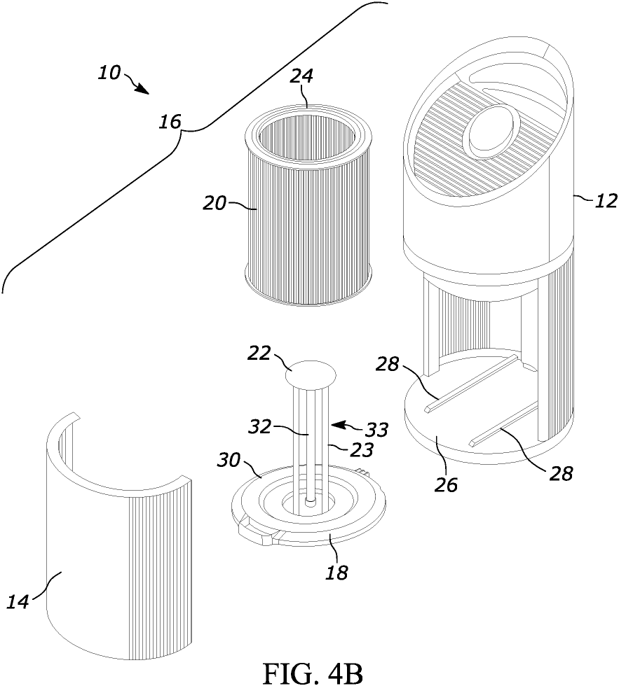

As shown in FIG. 7, the annular filter 20 includes an upper end 19 and a lower end 21 that engages the filter plate 18. The one or more UV light sources 32 substantially extends from the top surface of the filter plate 18 to adjacent the upper end of the annular filter 20, as shown in FIGS. 4A and 4B. In one illustrative embodiment, the UV light source 32 may substantially extend about 75% or more of the length between the filter base plate 18 and the upper end of the annular filter. The UV light source 32 may extend below the top surface 35 of the filter plate 18.

In other embodiments, as shown in FIG. 4B, a UV light source assembly 33 may be included as part of the filter assembly 16. In these embodiments, the annular filter 20 is removably disposed around the UV light source assembly 33. In one embodiment, the UV light source assembly 33 includes a stabilizer or bulb shield 22, the one or more UV light sources 32, and at least one supporting structure 23. The UV light source assembly 33, similar to the UV light source 32 discussed above, may be removably retained to the filter plate 18. The bulb shield 22 of the UV light source assembly 33 also may be retained to the filter plate 18. In one illustrative configuration, the UV light source 32 is held securely to the bulb shield 22. This may be accomplished through the use of threads on the bulb and corresponding threads on the bulb shield 22, or may be a push-fit utilizing friction to securely hold the UV light source 32. This may further be accomplished using a slotted cam mechanism on the UV light source 32 and bulb shield 22. The base of the UV light source 32 may be inserted into a cam slot on the bulb shield 22 and rotated to lock the UV light source 32 into place.

In one illustrative approach, a support structure 23 may be used to support the bulb shield 22. The support structure 23 may also be used to control the intensity of light emitted from the UV light source 32 onto the annular filter 20.

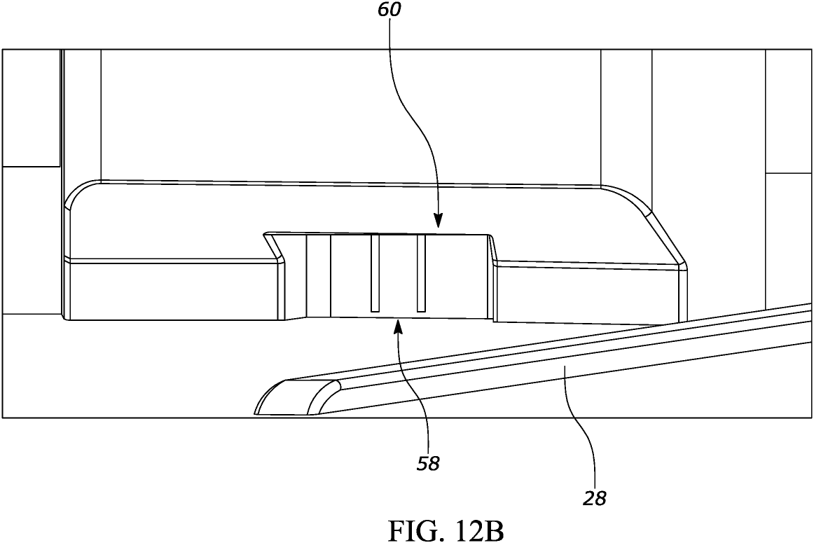

As shown, for example in FIGS. 12A and 12B, the air purifier 10 may also include a rear housing column 29. The rear housing column 29 is primarily disposed in the rear of the housing opposite the opening of the housing that permits insertion and removal of the filter assembly 16 from the interior of the housing. In some configurations, the rear housing column 29 provides structural support and or permits one or more portions of the filter assembly 16 to electrically engage a remainder of the housing. For example, the rear housing column 29 may electrically engage with the UV light source 32, described in detail below.

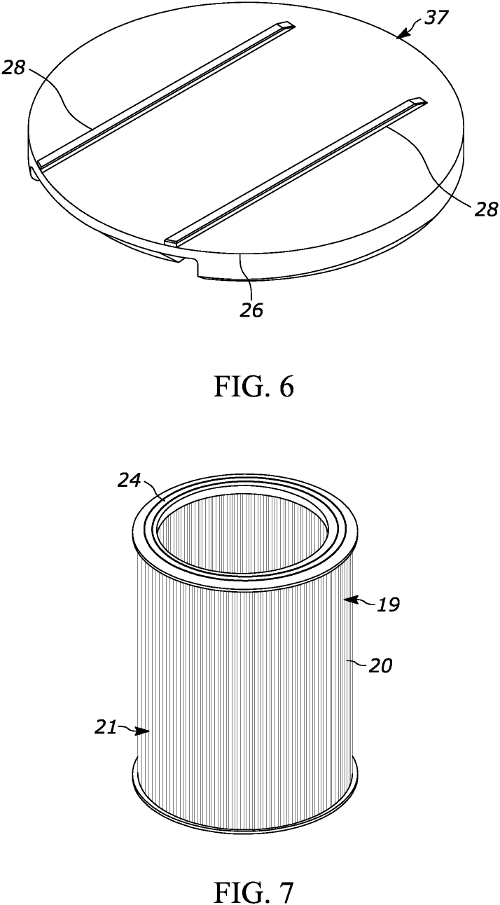

In the embodiment illustrated in FIGS. 5A and 6, the filter plate 18 includes a pair of slots 34 on the bottom surface 31 thereof. The base plate 26 includes a pair of guide rails 28 on the top surface 37 of the base plate 26. The guide rails 28 typically correspond to the geometry of the lower slots 34 of the filter plate 18. This may permit the filter plate 18 to horizontally slide from a first position outside of the housing 12 to a second position within the housing 12 through an opening in the housing, as the process is being illustrated in FIGS. 3A and 3B.

The filter plate 18 may include a grasping mechanism, such as a handle 36, on an edge thereof adjacent first ends of the pair of slots 34 that are disposed on the lower surface 31 of the filter plate 18, as shown in FIG. 2B. A user may grasp the handle 36 pull the filter plate, thereby horizontally sliding the filter plate 18 from the first position outside of the housing 12 to the second position within the housing 12, as shown in FIGS. 3A and 3B. This process may be inversely repeated with the user grasping the handle 36 and horizontally sliding the filter plate 18 from the second position within the housing 12 to the first position outside of the housing 12.

In use, the handle 36 may be accessible after the removable cover 14 is detached from the housing 12 to thereby expose an opening in a wall of the housing. A user may remove the removable cover 14, grasp the handle 36 through the opening, and slidably disconnect the filter plate 18 (including the filter assembly 16 from the base plate 26) from its seated position within the housing 12. This moves the filter plate 18 from the second position within or inside of the housing 12 to the first position outside of the housing 12. This may allow for a user to replace or clean the annular filter 20 and/or the UV light source 32, or other portions of the air purifier 10 that may require maintenance.

As shown in FIG. 5A, the filter plate 18 may include structure to facilitate receiving and retaining structures therein or thereon. For example, the filter plate 18, in some configurations, includes coupling structure that includes a plurality of grooves 30 on the upper surface 35 thereof. The plurality of grooves 30 are configured to retain at least the lower portion 21 of the annular filter 20, the UV light source 32, and/or the UV light source assembly 33 if used. The filter plate 18 may also include cavities, wells, or other depression to assist with coupling to or retaining one or more spring-loaded mechanisms 42, discussed in more detail below. The filter plate 18 may further include an opening 39 to allow for electrical components (or portions thereof) to pass between the bottom surface 31 of the filter plate 18 and the UV light source 32.

Referring to FIG. 7, the annular filter 20 includes the upper end 19 and the lower end 21. In some configurations, the annular filter 20 also includes a compressible ring 24. The compressible ring 24 may compress against a surface of an intermediate internal housing member 13. In some configurations, there may be one or more compressible rings 24 placed at upper end 19 and the lower end 21 of the annular filer 20. This allows for a substantially airtight seal between the annular filter 20 and the intermediate internal housing member 13. This means that the filter assembly 16 in the second position within the housing may create the substantially airtight seal such that when air is drawn into the air purifier 10, the air passes through the annular filter 20, past the UV light source 32, and enters the air blowing unit with minimal leakage of contaminated or unprocessed air entering the air blowing unit. The compressible ring 24 may be comprised of, for example, foam, plastic, rubber, and/or other suitable compressible materials. By some approaches the compressible ring provides a 360-degree area of compression between the annular filter 20 and an internal housing member 13 disposed above the filter. In one illustrative approach, the compressible ring 24 is comprised of, at least in part, a closed-cell foam to thereby provide a 360-degree compression. In one exemplary configuration, the compressible ring 24 provides 360-degrees of compression between an upper edge of the annular filter and the adjacent portion of the housing such that substantially no air, particles, contaminants, and/or other media can propagate through the compressible ring 24.

The substantially airtight seal between an upper portion of the annular filter 20 and an adjacent portion of the housing allows for minimal leakage between the annular filter 20, the compressible ring 24, and the intermediate internal housing member 13 (such that no air is moving into or out of the air purifier 10 at this junction). This may mean that the substantially airtight seal allows for at least 99% retention of air at the juncture of the annular filter 20, compressible ring 24, and intermediate internal housing member 13. The substantially airtight seal may aid the annular filter 20 in capturing at least 99% of all particulate matter larger than 0.1 microns. By some approaches, at least 99 % of all particles smaller than 3 microns are captured. In yet another approach, 99.97% of all particles smaller than 3 microns are captured. In other embodiments, the substantially airtight seal permits the annular filter 20 to capture at least 99% of all particles. In yet other embodiments, 99.95% of all particles are captured.

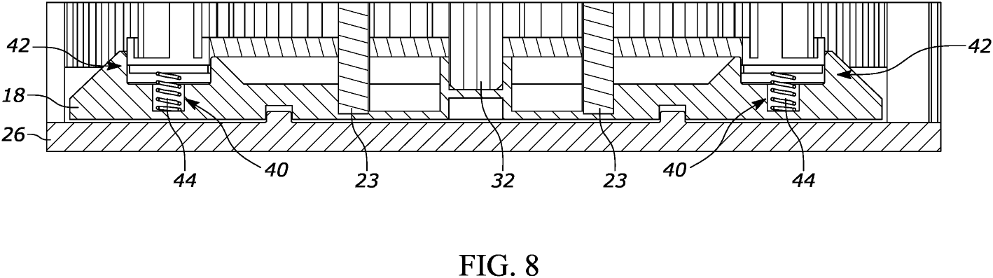

Referring to FIGS. 8 - 9C, the air purifier 10 may also include a spring-loaded mechanism 42. In some configurations the spring-loaded mechanism(s) 42 are housed inside of or on the top surface 35 of the filter plate 18. In operation, the spring-loaded mechanism 42 typically biases the annular filter 20 upward once the filter plate 18 is in a seated position, i.e., in the second position. In some configurations, a portion of the filter plate 18 may be upwardly biased with the annular filter 20. In some configurations, the spring-loaded mechanism 42 includes a filter plate ring 43 that contacts a bottom edge of the annular filter 20 to upwardly bias the annular filter 20. When the annular filter 20 is upwardly biased the compressible ring 24 of the upper end 19 of the annular filter 20 engages with the intermediate internal housing member 13. This may result in the substantially air-tight seal between the annular filter 20 and the intermediate internal housing member 13. The annular filter 20 may create the substantially airtight seal without the use of the compressible ring 24. In other configurations, the entire filter assembly 16 may be biased upward via one or more mechanisms. In yet other configurations, the orientation may be rotated such that the direction of the biasing is flipped. For example, the biasing member may be disposed atop the annular filter such that the filter is biased downward toward a compression member located at a lower end of the annular filter.

In one exemplary configuration, the spring-loaded mechanism 42 is disposed on the filter plate 18. The spring-loaded mechanism 42 may also be disposed inside of a portion of the filter plate 18. In some embodiments, the spring-loaded mechanism includes a plurality of cavities 40 and a plurality of springs 44 disposed therein. The plurality of cavities 40 may be covered by the filter plate ring 43 to conceal or keep the plurality of springs 44 inside of the cavities 40. The cavities 40 may also be wells, depressions, channels, or intrusions on or into the filter plate 18 to house the plurality of springs 44. A channel in the filter plate 18 may be used to allow for a compressible material to be disposed therein and provide the upward biasing force to the annular filter 20 to create the substantially airtight seal. In further embodiments a closed cell foam may be used in place of the springs to upwardly bias the annular filter to the intermediate internal housing member.

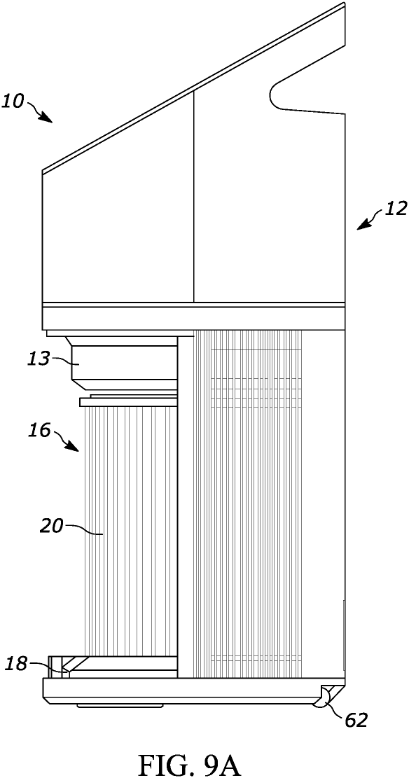

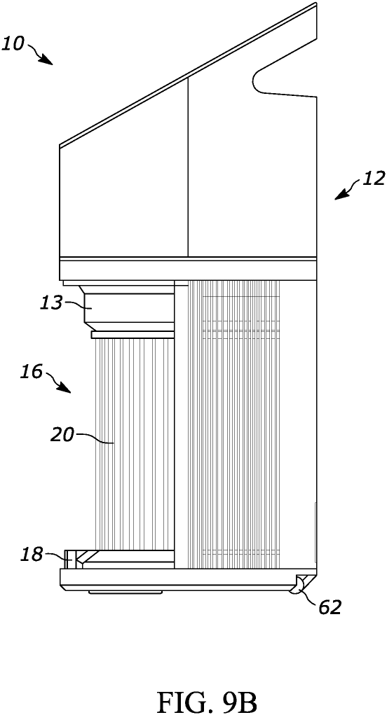

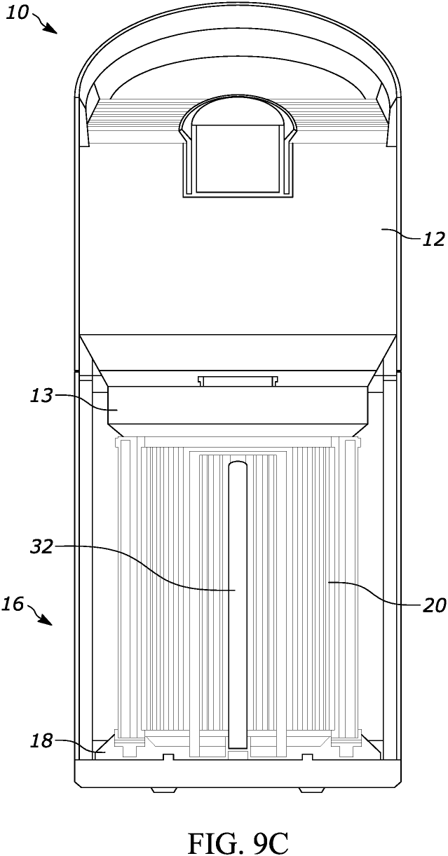

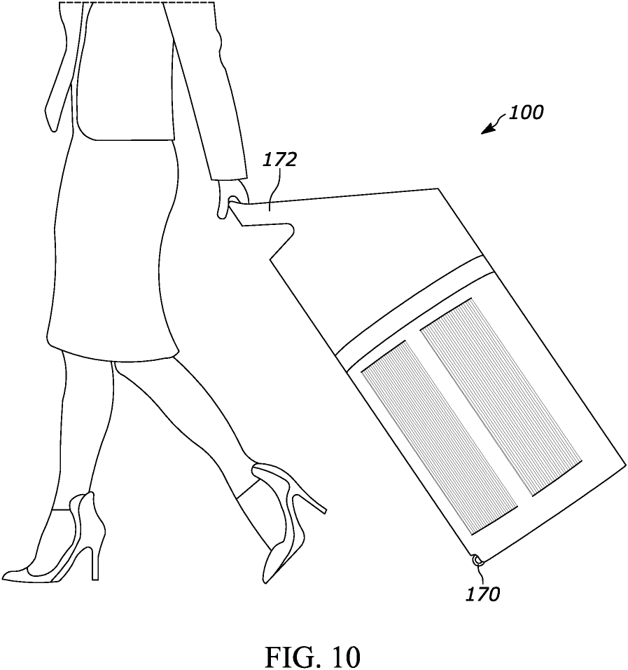

As shown in FIGS. 9A and 9B, once the filter plate 18 has slid into the second position, as shown in FIG. 9A, the annular filter 20 may be upwardly biased to create the substantially airtight seal, as shown in FIGS. 9B and 9C. FIG. 9A may be considered to illustrate the air purifier 10 with the filter plate 18 and annular filter 20 in an unseated position. An unseated position as defined herein is a position such that the annular filter 20 and/or the compressible ring 24 are not in full contact with the intermediate internal housing member 13. This means that when in an unseated position the annular filter 20, compressible ring 24, and intermediate internal housing member 13 do not create the substantially airtight seal.

FIG. 9B may be considered to illustrate the air purifier 10 with the filter plate 18 and annular filter 20 in a seated position. A seated position as defined herein is a position such that the air filter 20 and/or the compressible ring 24 are in contact with the intermediate internal housing member 13. This means that when in a seated position the annular filter 20, compressible ring 24, and intermediate housing member 13 create the substantially airtight seal.

As suggested above, in other embodiments, the spring-loaded mechanism 42 may be disposed on the intermediate internal housing member 13 and downwardly bias the annular filter 20 and filter plate 18 to create the substantially airtight seal.

In further embodiments a mechanical means may be used to press the annular filter into engagement with surrounding portions of the air purifier, such as by biasing the filter plate and annular filter to create the substantially airtight seal. Such embodiments may include the use of ramps and/or plateaus to create the substantially airtight seal. The ramps may be placed on the top surface of the base plate. Similar to the keyed connection described above, corresponding ramps may be disposed on the bottom surface of the filter plate such that the ramps of the base plate and filter plate align when the filter plate is moved to the second position within the housing. When the filter plate is moved to the second position the ramps of each plate align and upwardly bias the filter plate, annular filter, and compressible ring to the intermediate internal member creating the substantially airtight seal.

In further embodiments a rotational means may be used to upwardly bias the filter plate to create the substantially airtight seal. In these embodiments, the filter plate may include a secondary filter plate that is discretely rotatable relative to the filter plate. The secondary filter plate may include a protrusion that extends beyond an outer edge of the filter plate. In these embodiments, protrusions are disposed on the top surface of the base plate. Corresponding protrusions are disposed on a bottom surface of the secondary filter plate. The secondary filter plate may be rotatable once the filter plate has been inserted into the second position, or it may be rotatable at any time.

In such a configuration, in use, when the filter plate is inserted to the second position within the housing, the secondary filter plate may be rotated using the protrusion extending beyond the outer edge of the filter plate. When rotated, the secondary filter plate and corresponding protrusions rotate and engage the protrusions on the top surface of the base plate. Once engaged, the protrusions upwardly bias the filter plate, annular filter, and compressible ring to create the substantially airtight seal with the intermediate internal housing member.

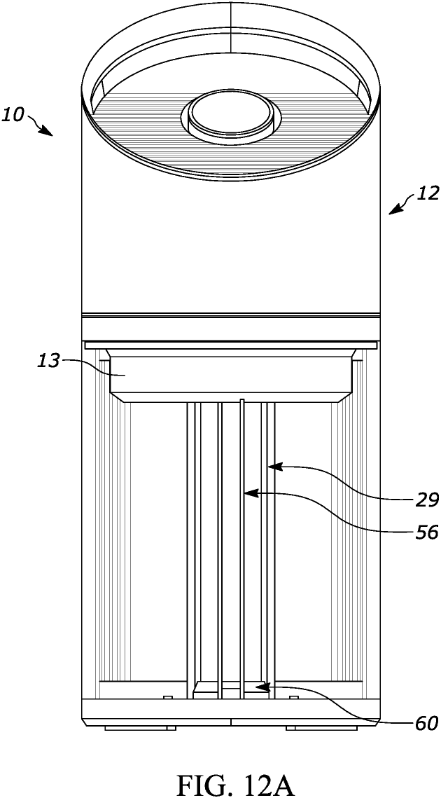

Referring now to FIGS. 11A and 11B, the air purifier 10 may include the filter plate 18 having a source electrical connector 50. The source electrical connector 50 may be on or adjacent to an outer edge of the filter plate 18. In one illustrative configuration, the source electrical connector 50 protrudes outward beyond the outer edge or rim of the filter plate 18. The source electrical connector 50 may electrically couple the UV light source 32 with the housing 12 via the filter plate 18 (or the electrically conductive connectors therein or thereon). The source electrical connector 50 includes at least one or more source electrical connections 52. The source electrical connections 52 are connected to source connection wire(s) 54. The source connection wire(s) 54 may be disposed under the filter plate 18 or inside of a channel of the filter plate 18. In addition, the source connection wire(s) 54 may connect to the UV light source 32. By one approach, the source connection wire(s) 54 couple to the UV light source 32 through an opening 39 of the filter plate 18. The source connection wires 54 may be disposed on, under, or through the filter plate 18 such that the wires are not in contact with the annular filter 20.

As illustrated in FIGS. 12A and 12B, the air purifier 10 may include a rear housing column 29 in the housing 12. In addition to providing structural support, the rear housing column 29 also may have a housing electrical connector 60. By one approach, the rear housing column 29 may have one or more housing connection wires 56 disposed therein. The housing electrical connector 60 includes at least one housing electrical connections 58. The housing electrical connections 58 may correspond to the source electrical connections 52 described above.

To provide power to the UV light source 32, the filter plate 18 may be horizontally inserted to the second position within the housing 12, the source electrical connector 50 may electrically couple with the housing 12 via a housing electrical connector 60. The electrical connection from the UV light source 32 and the housing 12 may follow the path of the UV light source 32 to the source connection wires 54, to the source electrical connections 52, to the housing electrical connections 58, to the housing connection wires 56, to the housing 12 which is connected to a power source. A power source may include a battery, wired connection, or other power source providing adequate current to the air purifier 10.

The source electrical connector 50 and housing electrical connector 60 may allow for the electrical connection to be severed when the filter plate 18 is moved from the second position. The disclosure also contemplates a kill-switch or other structure that disengages the source electrical connector 50 and housing electrical connector 60 when the removable cover 14 is removed from the housing 12. Other illustrative embodiments may include a sensor attached to the removable cover 14 to disengage the source electrical connector 50 and housing electrical connector 60. These sensors may include contact sensors or pressure sensors.

The electrical connection between the source electrical connector 50 and housing electrical connector 60 may be achieved through the use of, e.g., fingers, pads, plugs, magnetic connectors, pogo pins and/or other contact electrical connectors. These electrical connections, and other suitable electrical connections to couple the UV light source 32 to the housing 12 may be semi-permanent such that they allow for the severability between the electrical connection between the source electrical connector 50.

In some embodiments the source electrical connector 50 and the housing electrical connector 60 may function as a latching mechanism to latch the filter plate 18 to the housing 12 in addition to providing the electrical connection. This also may provide for a tactile confirmation of the filter plate 18 being seated properly inside the air purifier 10. Such a latching mechanism may allow for the movement or transportation of the air purifier 10 from place to place without concern that the filter plate 18 will become loose or the source and housing electrical connectors becoming disengaged. In some embodiments the source electrical connector 50 and the housing electrical connector 60 may include a magnet or sensor to confirm that the connectors are aligned and the UV light source 32 is ready to receive power. The sensor may send a signal to the user interface 8 to indicate that the connectors are aligned and ready to receive power.

In other embodiments a latching mechanism may be provided on or around the handle 36 of the filter plate 18 to provide a tactile or visual confirmation that the filter plate 18 is seated properly inside the housing 12.

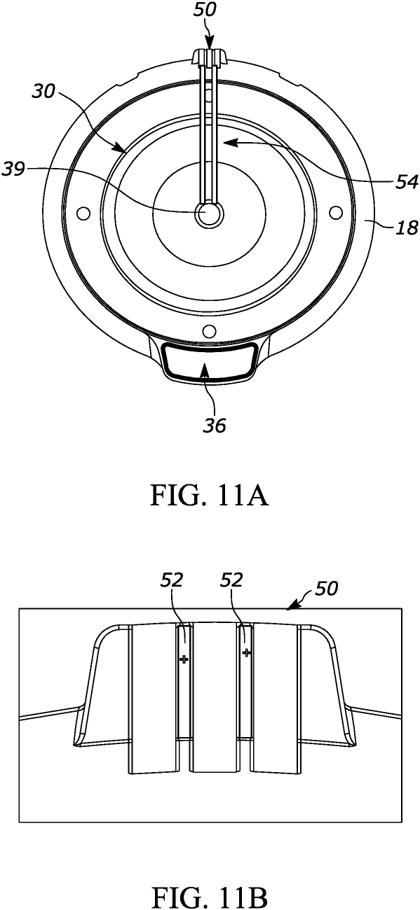

As shown in FIG. 10, the air purifiers provided herein also may be easily transported. In one illustrative configuration, an air purifier 100 includes one or more wheels 170 at an outside base thereof, thereby permitting a user to grasp a unit handle 172 and roll the air purifier from one location to another location.

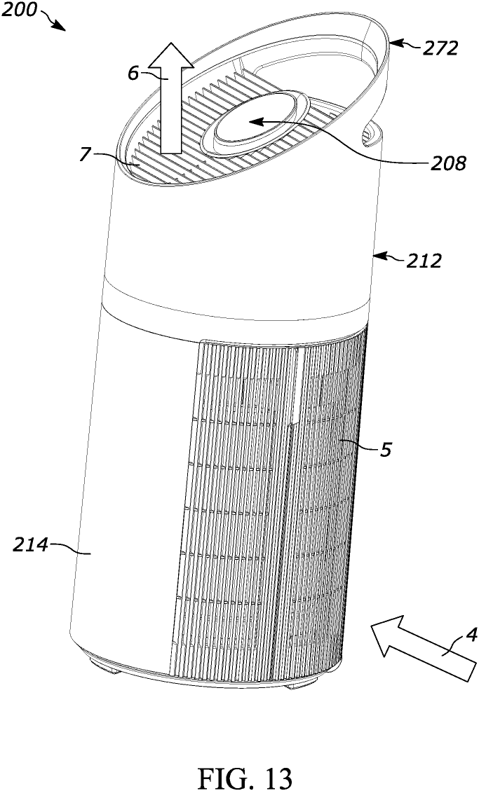

As shown in FIG. 13, an illustrative embodiment of the air purifier 200 is provided in accordance with some embodiments. The air purifier 200 is similar to the air purifier 10 discussed above. The differences between the air purifier 200 will be discussed below. These differences may be incorporated into the air purifier 10, and similarly, the elements associated with the air purifier 10 can be incorporated into the air purifier 200.

The air purifier 200, which operates like the air purifier 10, draws air in through an air intake 5 (as illustrated by arrow 4) on a side of the air purifier 200. Similarly, the air purifier 200 may process the air through a housing 212 (which contains an air filter assembly 216) and expels substantially purified air (as illustrated by arrow 6) at an air outlet 7 on a top of the air purifier 200. The air purifier 200 may also include a user interface 208 and a unit handle 272 that a user may grab to manipulate or move the air purifier 200.

As illustrated the air purifier 200 includes a housing 212 and a removable cover 214. An air blowing unit (as is known in the art) may be disposed within the housing 212. In one exemplary embodiment, the air purifier 200 includes a base plate 226 and a filter assembly 216. The filter assembly 216, described in more detail below, is removably disposed within the housing 212. In some embodiments, the filter assembly 216 includes a filter plate 218, an annular filter 220, a mounting plate 217, and one or more ultra-violet (UV) light sources 232 removably retained to the filter plate 218 and/or the mounting plate 217. The annular filter 220 is removably disposed around the ultra-violet light sources 232, also referred to as the UV source or UV light source. The filter plate 218 may further include a handle 236, described in more detail below.

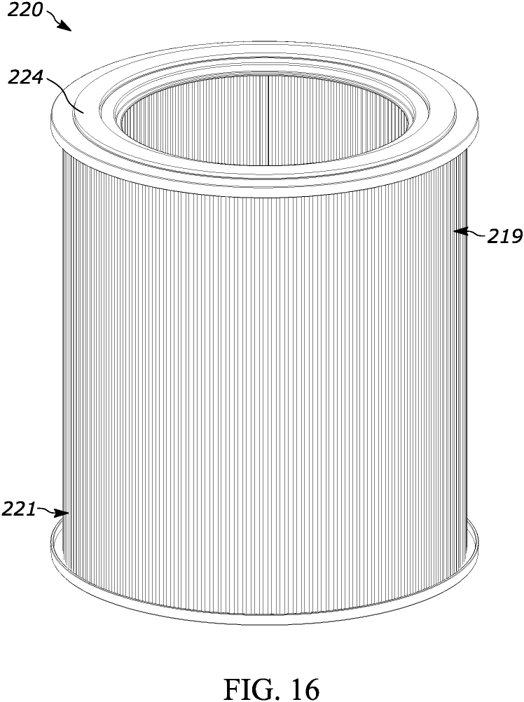

The annular filter 220, as shown in FIG. 16, similar to the annual filter 20 described in more detail above, includes the upper end 219 and the lower end 221. In some configurations, the annular filter 220 also includes a compressible ring 224. The annular filter 220 may include more than one compressible ring 224 at the top of the annular filter 220 and the bottom of the annular filter 220. The annular filter 220 having more than one compressible ring 224 allows a user to place the annular filter 220 in either configuration over the UV light source 232 while maintaining a substantially airtight seal. The compressible ring 224, similar to the compressible ring 24, may compress against a surface of an intermediate internal housing member 213 and/or the filter plate 218. The compressible ring 224 may be comprised of, for example, foam, plastic, rubber, and/or other suitable compressible materials. By some approaches the compressible ring provides a 360-degree area of compression between the annular filter 220 and an internal housing member 213 disposed above the filter. In one illustrative approach, the compressible ring 224 is comprised of, at least in part, a closed-cell foam to thereby provide a 360-degree compression. In one exemplary configuration, the compressible ring 224 at the top and the bottom of the annular filter 220 provides 360-degrees of compression between an upper edge of the annular filter 220 and the adjacent portion of the intermediate internal housing member 213 and between a lower edge of the annular filter 220 and a filter plate 218 such that substantially no air, particles, contaminants, and/or other media can propagate through and/or passed the compressible ring 224.

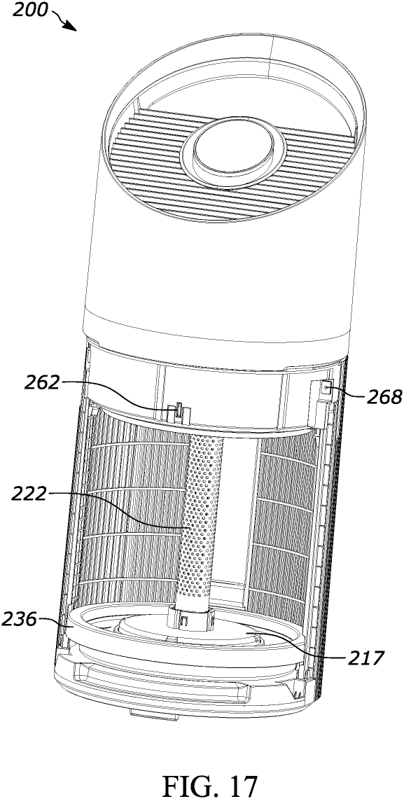

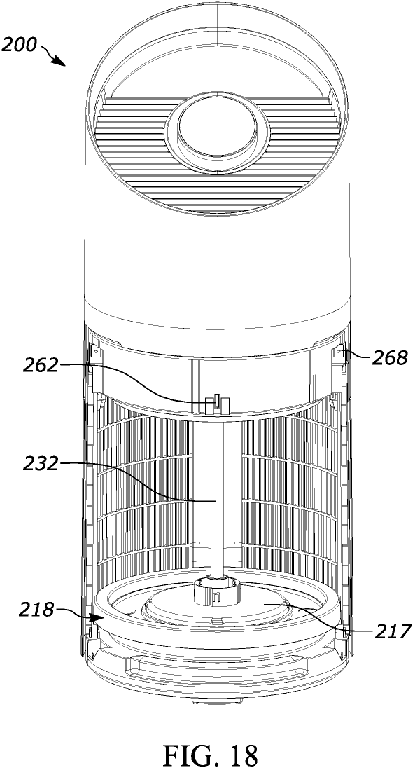

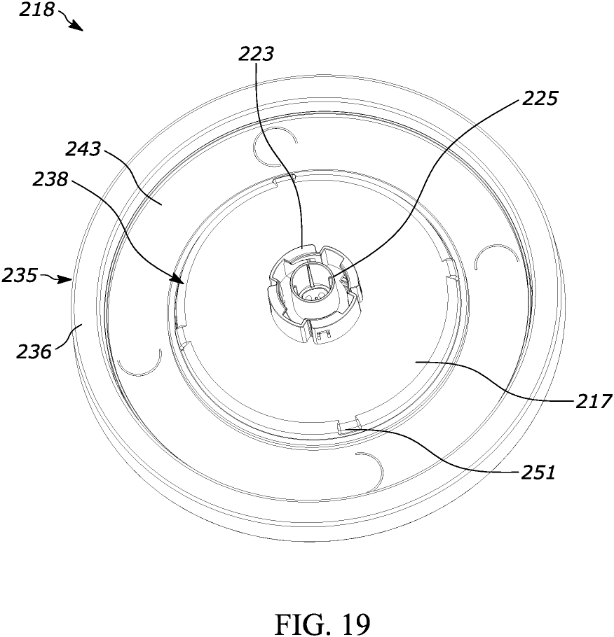

The mounting plate 217, as shown in FIGS. 17 to 19, is disposed on, coupled to, or integrally formed with the filter plate 218. The mounting plate 217 typically engages at least the filter plate 218, the annular filter 220, the UV light source 232, and the bulb shield 222. The mounting plate 217 further includes a socket 225 (FIG. 19) for the UV light source 232 to be disposed in. The socket 225 (as is known in the art) may provide added stability to the UV light source 232. The socket 225 may be a raised protrusion disposed on or extending from the center of the mounting plate 217. The UV light source 232 extends substantially the length of the annular filter 220. In one illustrative embodiment, the UV light source 232 may span about 50% to 100% of the length of the annular filter 220.

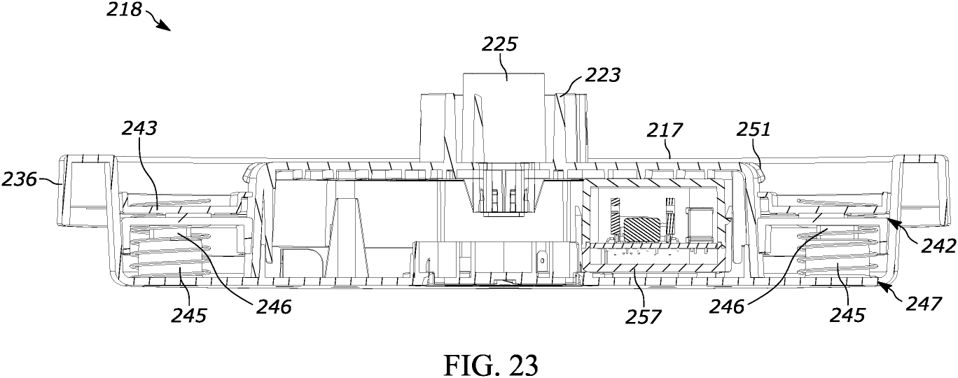

The mounting plate 217 may additionally provide shielding to electrical components housed inside of and/or beneath the mounting plate 217 and on the filter plate 218 from the UV light. A ballast 257, as shown in FIG. 23, for converting and or regulating the power to the UV light source 232, may be disposed beneath and/or within the mounting plate 217 and on the filter plate 218. Additionally, a bulb shield 222 may be disposed on, over, and/or around the UV light source 232 and engage the mounting plate 217 via a column holder 223.

The bulb shield 222 may be utilized to provide a more even distribution or irradiation of UV light across an inner surface of the annular filter 220. In this configuration, the bulb shield 222 may include reflective surfaces disposed on or around the bulb shield 222, either an inner surface or an outer surface of the bulb shield 222, to reflect, redirect, or distribute the UV light to the annular filter 220. Additionally or alternatively, the bulb shield 222 may include openings of the same or different sizes and shapes to provide more even distribution or irradiation of the UV light. Providing more even distribution of UV light across the annular filter 220 may allow for less degradation of an inner filter surface and provide more inactivation for particles and contaminants passing into and potentially through the annular filter 220. The bulb shield 222 may additionally protect, stabilize, and/or support the UV light source 232 disposed therein or thereon.

In one illustrative embodiment, as shown in FIG. 19, the mounting plate 217 includes a top surface with an edge 238. The top edge 238 includes one or more movable projections or tabs 251 disposed around the circumference to engage the annular filter 220. The annular filter 220 may be placed over the mounting plate 217 and held in place by the one or more tabs 251. By one approach, the tabs 251 are elastically deflectable and bias outward to engage the annular filter 220 when disposed around the edge 238 of the top surface. Because the mounting plate 217 is connected to and/or formed from the filter plate 218, the annular filter 220 is therefore connected to the filter plate 218 and held in place. The annular filter 220 is engaged by the tabs 251, the filter plate 218, specifically a filter plate ring 243, and the housing 212, specifically the internal housing member 213. Because the annular filter 220 is placed over the mounting plate 217, the mounting plate 217 and the annular filter 220 have corresponding sizes such that the annular filter 220 can snuggly fit over, around, or on the mounting plate 217 while maintaining the substantially airtight seal at the filter plate 218.

The mounting plate 217 additionally includes the socket 225 for the UV light source 232 and the column holder 223 for the bulb shield 222. In one exemplary embodiment, both the socket 225 and the column holder 223 extend from the top surface 235 of the mounting plate 217. The socket 225 and the column holder 223 may include engagement features to hold and/or stabilize the UV light source 232, corresponding to the socket 225, and the bulb shield 222, corresponding to the column holder 223. In one illustrative embodiment, the column holder 223 includes a raised an annular wall disposed around the socket 225. The socket 225 and the column holder 223 may be disposed on, coupled to, or integrally formed with the mounting plate 217.

Additionally, the air purifier 200 may have a mechanism that biases the filter plate 218 in a manner that facilitates engagement between the annular filter 220, the housing 212, and the filter plate 218. In one illustrative embodiment shown in FIG. 23, a spring-loaded mechanism 242 is provided on the filter plate 218. The spring-loaded mechanism 242 is disposed in, on, around, or otherwise coupled to the mounting plate 217 and the filter plate 218. By one approach, a filter plate ring 243 may be utilized as a component of the spring-loaded mechanism 242 for the annular filter 220 to be placed on or captured by. The spring-loaded mechanism 242, similar to that described above with reference to the spring-loaded mechanism 42, includes a plurality of springs. In some configurations one or more compressible elastic elements may be used in the spring-loaded mechanism 242. For example, a washer spring, leaf spring, or a coil spring may be used. In some configurations, three or more spring may be used in the spring-loaded mechanism 242. In one illustrative embodiment, four springs are used in the spring-loaded mechanism 242.

In one illustrative embodiment, the filter plate ring 243 is coupled to the spring-loaded mechanism 242 and is disposed around the mounting plate 217. Additionally, the filter plate 218 includes a bottom surface channel 247 which is disposed around the mounting plate 217. The plurality of springs are disposed between the bottom surface channel 247 and the spring-loaded mechanism 242 and/or the filter plate ring 243. Additionally, the plurality of springs are disposed on, around, or over posts 245. In this configuration, there are a corresponding number of posts and springs used in the spring-loaded mechanism 242 to aid in facilitating the upward biasing of the spring-loaded mechanism 242, the filter plate ring 243, and the annular filter 220. The posts 245 are disposed on, coupled to, or integrally formed with the filter plate 218 and extend upward from the filter plate 218 towards the filter plate ring 243. The posts 245 are sized to allow for the compression of the plurality of springs to allow for the annular filter 220 to be properly seated inside the air purifier 200 while creating and maintaining the substantially airtight seal.

Additionally, the posts 245 include openings for filter plate ring columns 246. The filer plate ring columns 246 are coupled to the filter plate ring 243. The filter plate ring columns 246 allows the filter plate ring 243, and in turn, the spring-loaded mechanism 242, to be connected to the filter plate 218 using snaps that connect to the posts 245. Once the filter plate ring columns 246 are connected to the posts 245, the spring-loaded mechanism 242 is coupled to the filter plate 218 prohibiting the springs from coming out of the spring-loaded mechanism 242. Similar to the posts 245, the filter plate ring columns 246 have a corresponding filter plate ring column 246 for each spring, and additionally are sized to allow for the compression of the plurality of spring to allow for the annular filter 220 to be properly seated inside the air purifier 200 while creating and maintaining the substantially airtight seal.

As described above, the spring-loaded mechanism 242 biases the annular filter 220 upward against an intermediate internal housing member 213. In one illustrative embodiment the annular filter 220 includes a compressible ring 224 at both the top and the bottom of the annular filter 220 to allow a user to place either side of the annular filter 220 over the UV light source 232. In this configuration, the annular filter 220 has a compressible ring 224 contacting the intermediate internal housing member 213 and a second compressible ring contacting the filter plate 218, and more specifically the filter plate ring 243.

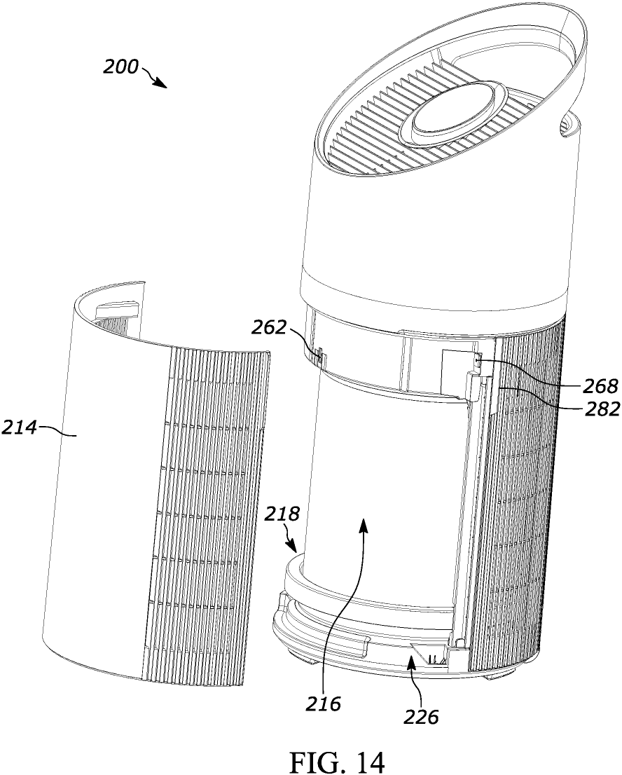

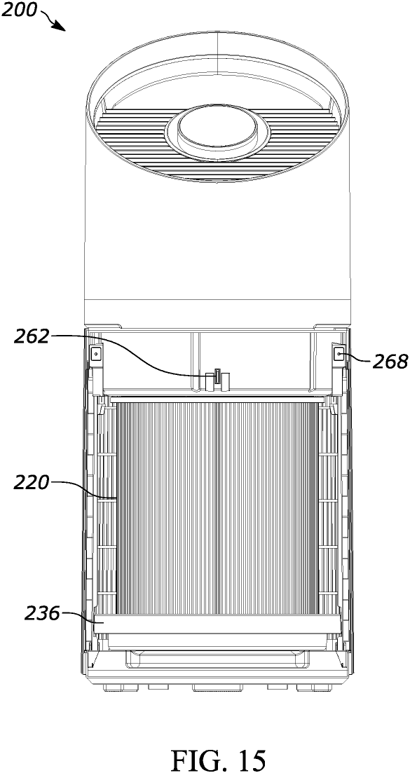

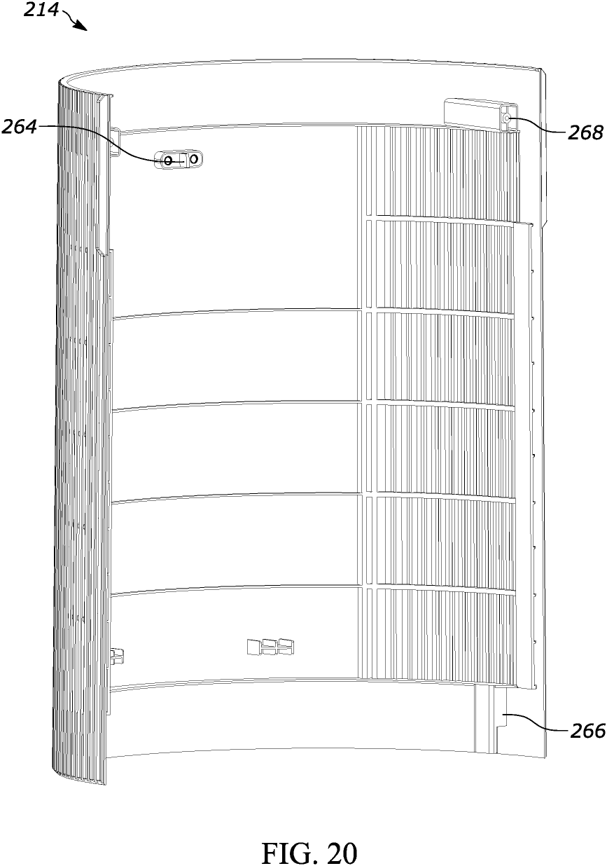

In one illustrative embodiment, as shown in FIGS. 14, 15, 17, and 18, the air purifier 200 includes one or more engagement mechanisms, such as an interlock or interlocking device 262. The interlocks 262 may be functionally utilized as a switch to control or permit the flow of electricity, from a power source or power supply, to the air purifier 200. In one illustrative embodiment the flow of electricity passes from the power supply to a base plate 226 and/or the housing 212, the filter plate 218, the ballast 257, the mounting plate 217, and the one or more UV light sources 232. In one approach, the interlocks 262 have corresponding interlock tabs 264 that engage the interlock 262 and allow for the flow of electricity, as shown in FIG. 20. The interlocks 262 may be used to prevent a user from being exposed to the UV light, and in some instances, bright UV light from the UV light source 232. Additionally, the interlocks 262 may be disposed such that a user cannot engage the interlocks 262 with their finger to avoid the potential accidental exposure.

The interlocks 262 may be disposed on and/or inside the housing 212 to be engaged by the interlock tabs 264. The interlock tabs 264 may be protrusions disposed on the removable cover 214, the filter plate 218, the filter 220, or any other movable feature to engage the interlocks 262. In one embodiment, as shown in FIG. 20, the removable cover 214 includes at least one interlock tab 264. The interlock tab 264 corresponds to an interlock 262 disposed within and/or on the housing 212 or the intermediate internal housing member 213. When the removable cover 214 is placed on the housing 212, the interlock tab 264 engages the interlock 262 and permits the flow of electricity such that the UV light source 232 is permitted to turn on. Additionally or alternatively, an interlock 262 (or a portion thereof) may be placed on the filter plate 218 such that when the filter plate 218 is fully inserted into the housing 212, electricity may be permitted to flow. In one illustrative embodiment, the intermediate internal housing member 213 may include both the interlock 262 and the interlock tab 264. In this embodiment, the interlock tab 264 engages the interlock 262 once the annular filter 220 is biased upward by the spring-loaded mechanism 242. This configuration may be accomplished using a rotation structure such as a plunger rotating by the insertion of the annular filter 220 and then engages the interlock 262 by the upward biasing. Additionally or alternatively, the interlock 262 may engage the interlock tab 264 as the annular filter 220 is being inserted into the housing 212.

In one illustrative embodiment, as shown in FIG. 20, the removable cover 214 includes one or more pivot features 266. The pivot features 266 include corresponding locations disposed on the housing 212 and/or the base plate 226 to be disposed in when assembled. The pivot features 266 may provide for an easier removal of the removable cover 214 from the housing 212. The removable cover 214 and the housing 212 may include magnets 268 to secure the removable cover 214 to the housing 212. The interlocks 262, interlock tabs 264, pivot features 266, and the magnets 268 may be disposed along any portion of the housing 212 and the removable cover 214 in corresponding locations, such that when the removable cover 214 is placed on the housing 212 the above features align. In one illustrative approach, one or more of these features are disposed adjacent an edge or periphery of one or both of the removable cover 214 or opening within the housing 212 that receives or engage with the removable cover 214. The magnets 268 described herein may include magnets disposed on both the housing 212 and the removable cover 214, or include a magnet placed on one of the above stated surfaces with the other surface having a corresponding magnet surface to magnetically couple the two surfaces together.

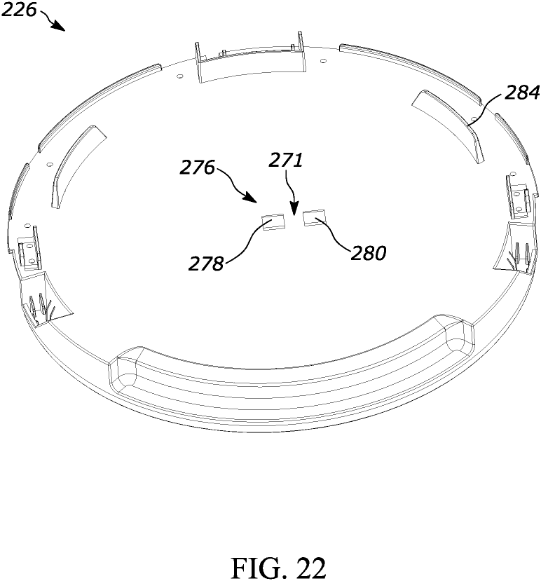

In some configurations, the base plate 226, as shown in FIG. 22, includes alignment features 284. By one approach, the alignment features 284 are used to guide and/or ensure the filter plate 218 is fully and properly inserted into the housing 212. The alignment features 284 may additionally ensure there is proper alignment for the electrical connections 276 and the electrically conductive pads 270, discussed in more detail below. By ensuring the proper alignment of the filter plate 218, and in turn the annular filter 220, the alignment features 284 may ensure the substantially airtight seal and the alignment of the interlocks 262 and interlock tabs 264.

In one illustrative embodiment the filter plate 218 includes a grasping feature, such as a handle 236. As shown in FIG. 19, the handle 236 may be formed or created by a substantially continuous lip around a circumferential top surface 235 of the filter plate 218. The handle 236 allows for a user to grasp the handle 236 around any portion of the filter plate 218 for easy removal of the filter assembly 216. The lip forming the handle 236 may be an undercut or an upward protrusion that spans the circumference/perimeter of the top surface 235 of the filter plate 218.

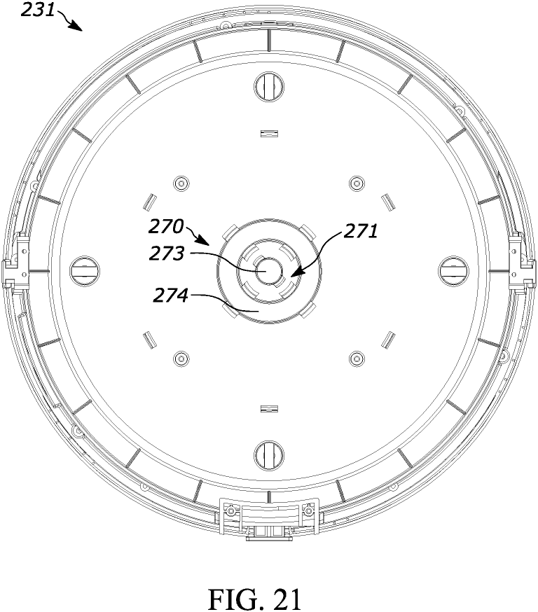

Referring now to FIG. 21, a bottom surface 231 of the filter plate 218 is illustrated. The bottom surface 231 may include one or more electrically conductive pads 270 disposed on the bottom surface 231 of the filter plate 218. The electrically conductive pads 270 may be comprised of, for example, one or more pieces of metal. In one illustrative embodiment there may be two electrically conductive pads, or pads, which may be disposed at different locations on the base or bottom. By some approaches, one of the pads may be a center pad 273 disposed substantially in the middle of the bottom surface 231. In one illustrative embodiment the center pad 273 may be square shaped. The second pad may a ring-shaped pad 274 disposed around the center pad 273. In this illustrative embodiment there is a gap 271 between the center pad 273 and the ring-shaped pad 274. The pads 273 and 274 may be electrically coupled to the ballast 257. The ballast 257 may be used, as stated above, to transmit the electricity provide by the power supply to the UV light source 232.

As shown in FIG. 22, the base plate 226 typically includes at least one electrical connection 276. The electrical connections 276 may be, for example, leaf springs. The electrical connections 276 may be disposed on the base plate 226 such that they are substantially side-by-side as compared to the insertion direction of the filter 220. Specifically, in some embodiments, one of the electrical connections 276 may be a center-aligned leaf spring 278 such that it is disposed in substantially the middle of the base plate 226. Further, a second leaf spring 280 may be disposed next to the center-aligned leaf spring 278. The electrical connections 276 may be disposed the same distance apart as the gap 271 of the conductive pads 270. Because the spacing between the electrical connections 276 and the one or more electrically conductive pads 270 are the same, and due to the shape of the ring-shaped pad 274, a user can insert the filter plate 218, and in turn the annular filter 220, at any rotation about a centerline running through the UV light source 232 and complete the electrical circuit.

In some approaches, when the filter plate 218 is inserted into the housing 212, the filter plate 218 biases the electrical connections 276 downward completing the circuit between the UV light source 232 and the power supply. The downward biasing of the electrical connections 276 may be due to the weight of the filter assembly 216 and/or the spring-loaded mechanism 242 biasing the filter 220 against the intermediate internal housing member 213 and forcing the filter plate 218 downward.

In use, as a method for the removal and/or the replacement of the filter 220 or the UV light source 232, a user grasps the removable cover 214 at one or more finger indentations 282 placed on the side of the housing 212 near the removable cover 214 and separates the removable cover 214 from a remainder of the housing 212. A user typically removes the removable cover 214 by overcoming strength of the magnets 268 disposed on the removable cover 214 and the housing 212. In some configurations, the removable cover 214 pivots about the pivot feature 266 at the bottom of the removable cover 214. After the removal of the removable cover 214, in embodiments utilizing interlock tabs 264 on the removable cover 214, the interlock 262 is disengaged by the interlock tab 264, and thus stopping the flow of electricity to the UV light source 232.

Further, a user may grasp the handle 236 of the filter plate 218 and manually remove or pulls the filter assembly 216 from an interior of the body of the air purifier 200. In doing so, the electrical circuit between the power supply and the UV light source 232 is severed. Once the filter assembly 216 is removed, a user has substantial access to the annular filter 220 and the light source 232. Indeed, a user may now easily remove the annular filter 220 from the filter plate 218 and clean or replace the annular filter 220. Similarly, for the replacement of the UV light source 232, the user removes the bulb shield 222 and then is permitted to remove the UV light source 232 from the top surface 235 of the mounting plate 217.

Those skilled in the art will recognize that a wide variety of modifications, alterations, and combinations can be made with respect to the above-described embodiments without departing from the scope of the disclosure, and that such modifications, alterations, and combinations are to be viewed as being within the ambit of the disclosed concept.

What is claimed:

1. An air purifier comprising:
a housing having a base plate and a removable cover;
an air blowing unit disposed within the housing; and
a filter assembly removably disposed within the housing, the filter assembly including a filter plate, an annular filter, a mounting plate, and one or more ultra-violet (UV) light sources removably retained to at least one of the filter plate and the mounting plate, wherein the annular filter is removably disposed around the one or more UV light sources;
wherein the filter plate slidably engages with the base plate of the housing; and
wherein the annular filter includes an upper end and a lower end, wherein the lower end engages the mounting plate, and the upper end that engages an intermediate, and
wherein the one or more UV light sources substantially extends from the filter plate to adjacent the upper end of the annular filter.

2. The air purifier of claim 1 wherein the mounting plate is configured to engage the annular filter, the one or more UV light sources, and the filter plate, wherein the filter plate or the base plate includes two electrically conductive pads and two electrical connections, and wherein the annular filter further comprises one or more compression rings.

3. The air purifier of claim 1 further comprising an electrically conductive path between the housing, the filter plate, the mounting plate, and the one or more UV light sources.

4. The air purifier of claim 3 further comprising at least one interlock disposed on the housing, wherein the interlock permits a flow of electricity through the electrically conductive path once the interlock is engaged by a corresponding interlock tab disposed on at least one of the removable cover and the filter plate.

5. The air purifier of claim 1 further comprising at least one of a spring-loaded mechanism that biases the annular filter upward once the filter plate is seated within the housing, thereby pushing the upper end of the annular filter into engagement with an intermediate internal housing member.

6. The air purifier of claim 5 wherein the spring-loaded mechanism is disposed on the filter plate and biases the annular filter upward into engagement with the intermediate internal housing member once the filter plate is seated within the housing.

7. The air purifier of claim 5 wherein the filter plate further includes a plurality of grooves on an upper surface thereof, wherein the plurality of grooves are configured to retain at least one of the spring-loaded mechanism, the mounting plate, a lower portion of the annular filter, and a UV source assembly.

8. The air purifier of claim 1 wherein the housing further comprises an air inlet disposed along at least a portion of a side of the housing and an air outlet disposed along a portion of an upper surface of the housing.

9. A method for horizontal removal for an air purifier comprising;
removing a removable cover from a housing, wherein the housing includes a base plate and an air blowing unit;
engaging a handle disposed on a filter plate;
removing a filter assembly, wherein the filter assembly is disposed within the housing, and the filter assembly including a filter plate, an annular filter, a mounting plate, and one or more ultra-violet (UV) light sources removably retained to at least one of the filter plate and the mounting plate, wherein the annular filter is removably disposed around the one or more UV light sources; and
removing at least one of the annular filter and the UV light source for inspection, cleaning, or replacement.

10. The method of claim 9 wherein the mounting plate is configured to engage the annular filter, the one or more UV light sources, and the filter plate.

11. The method of claim 9 wherein the filter plate further comprises a grasping handle on an edge disposed around an upper surface of the filter plate, wherein the grasping handle is accessible after the removable cover is detached from a remainder of the housing.

12. The method of claim 9 further comprising an electrically conductive path between the housing, the filter plate, the mounting plate, and the one or more UV light sources.

13. The method of claim 12 further comprising at least one interlock disposed on the housing, wherein the interlock permits a flow of electricity through the electrically conductive path once the interlock is engaged by a corresponding interlock tab disposed on at least one of the removable cover and the filter plate.

14. The method of claim 9 further comprising at least one of a spring-loaded mechanism that biases the annular filter upward once the filter plate is seated within the housing, thereby pushing an upper end of the annular filter into engagement with an intermediate internal housing member.

15. The method of claim 14 wherein the spring-loaded mechanism is disposed on the filter plate and biases the annular filter upward into engagement with the intermediate internal housing member once the filter plate is seated within the housing.

16. The method of claim 14 wherein the filter plate further includes a plurality of grooves on an upper surface thereof, wherein the plurality of grooves are configured to retain at least one of the spring-loaded mechanism, the mounting plate, a lower portion of the annular filter, and a UV source assembly.

17. The air purifier of claim 9 wherein the filter plate includes two electrically conductive pads.

18. The air purifier of claim 9 wherein the base plate includes two electrical connections.

19. The method of claim 9 wherein the housing further comprises an air inlet disposed along at least a portion of a side of the housing and an air outlet disposed along a portion of an upper surface of the housing.

20. An air purifier comprising:
a housing having a base plate and a removable cover;
an air blowing unit disposed within the housing; and
a filter assembly removably disposed within the housing, the filter assembly including a filter plate, an annular filter, a mounting plate, and one or more ultra-violet (UV) light sources removably retained to at least one of the filter plate and the mounting plate, wherein the annular filter is removably disposed around the one or more UV light sources;
wherein the filter plate includes electrically conductive pads;
wherein the base plate includes electrical connections which correspond to the electrically conductive pads; and
wherein the annular filter includes an upper end and a lower end that engages the mounting plate, and wherein the one or more UV light sources substantially extend from the filter plate to adjacent the upper end of the annular filter.
